(19) United States
(12) Patent Application Publication
(10) Pub. No.: US 2015/0000837 A1
(43) Pub. Date: Jan. 1, 2015

(54) METHOD OF MANUFACTURING FINGERPRINT RECOGNITION HOME KEY FOR IMPROVING ATTACHMENT PRECISION OF FINGERPRINT RECOGNITION SENSOR

(71) Applicant: DREAMTECH CO., LTD, Seongnam-si (KR)

(72) Inventor: Young-Ho KIM, Yongin-si (KR)

(21) Appl. No.: 14/314,290

(22) Filed: Jun. 25, 2014

(30) Foreign Application Priority Data
Jul. 1, 2013 (KR) 10-2013-0076583

(51) Int. Cl. A61B 5/117

(52) U.S. Cl. CPC A61B 5/1172
USPC 156/275.5

(57) ABSTRACT

A method of manufacturing a fingerprint recognition home key for improving an attachment precision of a fingerprint recognition sensor in accordance with an embodiment of the present invention comprising the steps of an injection step in which a transparent injection molding product equipping a receiving groove, on which a fingerprint recognition sensor is placed, is molded, coating a UV curable bond on the transparent injection molding product, and adjoining the transparent injection molding product and a fingerprint recognition sensor using the UV curable bond is provided.

### CROSS-REFERENCE TO RELATED APPLICATION

This application claims priority to Korean Patent Application No. 10-2013-0076583 filed on Jul. 1, 2013, and all the benefits accruing therefrom under 35 U.S.C. §119, the contents of which is incorporated by reference in its entirety.

### BACKGROUND

1. Technical Field

An embodiment of the present invention relates to a method of manufacturing a fingerprint recognition home key for improving an attachment precision of a fingerprint recognition sensor able to adjust so that a liquefied bond may be coated in a constant thickness by using a UV curable bond on a junction between a fingerprint recognition sensor and a transparent injection molding product, while molding an injection molding product, on which a fingerprint recognition sensor is attached, transparently.

2. Description of the Related Art

A fingerprint recognition technology is a technology used to prevent various security accidents by forcing to go through user registrations and authorization procedures. For a detailed example, it is used for defense of individual and organizational networks, protection of various contents and data, safe access control, etc.

Recently, in accordance with sudden increase in users of various handheld devices comprising smartphones, tablet PCs, etc., accidents where personal information and contents stored in handheld devices, unintended by users, are leaked outside.

FIG. 1 is a drawing showing two typical examples of normal handheld devices.

FIG. 1 (a) is a smartphone, and FIG. 1 (b) is a tablet PC. Referring to FIG. 1 (a) and (b) together, a smartphone (10) or a tablet PC (20) is composed of a main body (11) (12), a large-area display device (13) (23) equipped in the front center of each main body, and a home key (H) equipped on one side of each display device.

The home key (H) is a configuration equipped to realize preset operations in the smartphone or the tablet PC, and provides convenient functions such as returning to an initial screen of the device when the home key (H) is pressed or touched when using the handheld device.

A capacitive method fingerprint recognition technology is a method reading unique shapes of different fingerprints of each user as an electrical signal by using electrical conductive characteristics of skins. The configuration of the home key with the fingerprint recognition sensor of this method built in may be identified by referring to FIG. 2.

FIG. 2 is a cross sectional drawing of a fingerprint recognition home key. The fingerprint recognition sensor is built in on top of a product (M), which is provided in a home key shape by methods of injection molding, etc.

The fingerprint recognition sensor equips an ASIC (110a) and a FPCB film (110b), and the ASIC (110a) takes the role of changing the detected analog data to a digital signal and is electrically connected to the FPCB film (110b), on which a pattern where fingerprints are recognized is formed.

And on top of this, a base coating layer by primer coating and shielding layer forming, a middle coating layer by a process of painting, etc., and a top coating layer by a UV coating treatment are formed in sequence, and a coating part (c) having a fixed thickness is formed. But if the thickness of this coating part (C) does not satisfy within a preset criterion and becomes thicker than this, problems of the fingerprint recognition worsening may occur.

Meanwhile, with respect to manufacturing a home key equipping the fingerprint recognition sensor, a coating method of an adhesive for the fingerprint recognition sensor to be attached mostly used a bond in a liquefied form. But, the bond in the liquefied form may incline to a certain direction unintended by a worker after coating due to the high liquidity. If the bond in the liquefied form is not coated uniformly, this has been the reason for the fingerprint recognition degrading due to a specific portion forming relatively thicker.

Therefore, there are needs for improving fingerprint recognition of fingerprint recognition home keys for improving these types of problems.

Korea laid-open patent publication No. 2002-0016671 (published 2002 Mar. 2006) is a prior art related to the present invention, and in the prior publication, a handheld information device with a fingerprint recognition module built in, and a technology related to its control method is disclosed.

### BRIEF SUMMARY

A method of manufacturing a fingerprint recognition home key for improving an attachment precision of a fingerprint recognition sensor able to improve fingerprint recognition by not only adjusting an amount of bond coated on an adjoining area of a fingerprint recognition sensor, but also aligning so that the coated bond has a uniform thickness is provided.

The problem the present invention intends to solve is not limited to the above described problems, and other problems not mentioned here will be apparent to those in the arts from the following disclosures.

A method of manufacturing a fingerprint recognition home key for improving an attachment precision of a fingerprint recognition sensor in accordance with an embodiment of the present invention comprises the steps of: (a) an injection step in which a transparent injection molding product equipping a receiving groove on which a fingerprint recognition sensor is placed is molded; (b) coating a UV curable bond on the transparent injection molding product; and (c) adjoining the transparent injection molding product and a fingerprint recognition sensor using the UV curable bond.

In the (a) step, an injection molding material composed of a transparent material comprising at least one among PC, PC glass, ABS, PP, PET, nylon, and plastic polymer compound may be used.

In the (b) step, aligning a coating state of the UV curable bond uniformly may be further comprised.

In the (c) step, (c-1) drying the UV curable bond ; and (c-2) performing a UV exposure to the dried UV curable bond ; may be further comprised.

Also, a method of manufacturing a fingerprint recognition home key for improving an attachment precision of a fingerprint recognition sensor in accordance with an embodiment of the present invention comprises the steps of: (a) a first injection step in which a transparent injection molding product equipping a receiving groove on which a fingerprint recognition sensor is placed is molded; (b) coating a UV curable bond on the transparent injection molding product; (c) adjoining the transparent injection molding product and a fingerprint recognition sensor using the UV curable bond; (d) a second injection step in which a second injection molding product in a shape of a home key equipping the adjoined fingerprint recognition sensor; (e) coating an adhesive or a pressure-sensitive material on a film of the fingerprint recognition sensor; and (0 adjoining a prepared exterior member on a film of the adhesive or pressure-sensitive adhesive material coated fingerprint recognition sensor.

In the (a) step, an injection molding material composed of a transparent material comprising at least one among PC, PC glass, ABS, PP, PET, nylon, and plastic polymer compound may be used.

In the (b) step, aligning a coating state of the UV curable bond uniformly may be further comprised.

In the (c) step, (c-1) drying the UV curable bond; and (c-2) performing a UV exposure to the dried UV curable bond; may be further comprised.

In the (e) step, a method coating a pressure-sensitive adhesive or an adhesive, or attaching a double-sided tape on the film of the fingerprint recognition sensor may be used. Before the (f) step, printing selected colors at least once on a back side of the exterior member after deposition.

The exterior member may be composed of at least one material among a normal glass, a sapphire glass, a plastic film or a thin film injection molding product.

In accordance with an embodiment of the present invention, a fingerprint recognition home key, in which a fingerprint recognition function of users may be realized by being equipped in various handheld devices, may be provided.

In accordance with an embodiment of the present invention, not only an amount of adhesive coated on an adjoining area of a fingerprint recognition sensor may be adjusted, but also may be aligned so that the coated bond may have a uniform thickness. Accordingly, the fingerprint recognition of the fingerprint recognition home key may be improved.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is a drawing illustrating an outline of a handheld phone and a tablet PC for detailed examples of typical handheld devices.

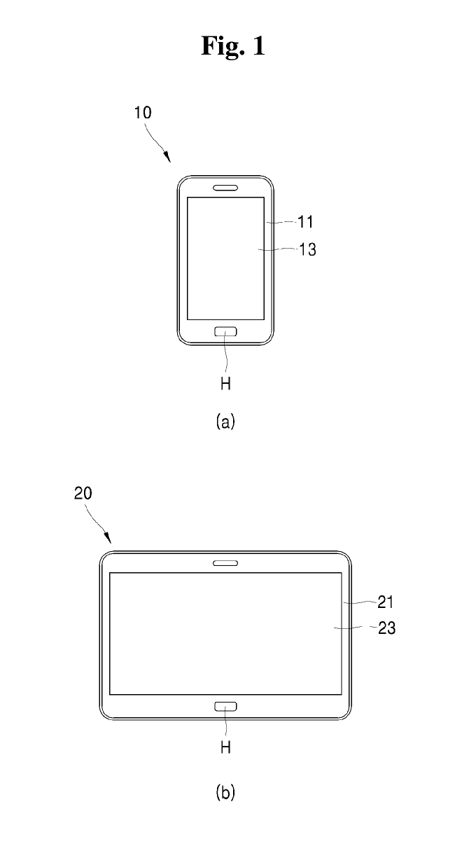

FIG. 2 is a drawing illustrating an outline of an internal structure of a fingerprint recognition home key.

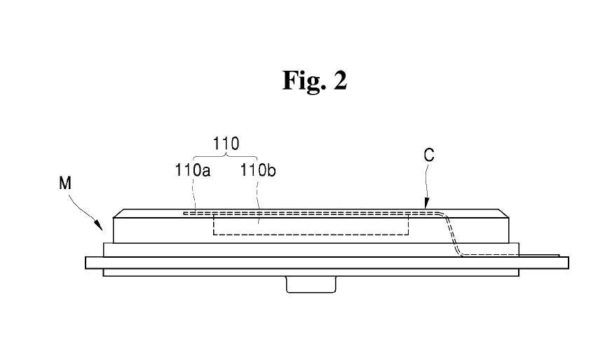

FIG. 3 is a flow chart of a method of manufacturing a fingerprint recognition home key for improving an attachment precision of a fingerprint recognition sensor in accordance with a first embodiment of the present invention.

FIG. 4 is a flow chart showing a detailed sequence of an adjoining step using a UV curable bond in the first embodiment of the present invention.

FIG. 5 is a process chart of a method of manufacturing a fingerprint recognition home key for improving an attachment precision of a fingerprint recognition sensor in accordance with a first embodiment of the present invention.

FIG. 6 is a flow chart of a method of manufacturing a fingerprint recognition home key for improving an attachment precision of a fingerprint recognition sensor in accordance with a second embodiment of the present invention.

FIG. 7 is a process chart of a method of manufacturing a fingerprint recognition home key for improving an attachment precision of a fingerprint recognition sensor in accordance with a second embodiment of the present invention.

### DETAILED DESCRIPTION

The terms and vocabulary used in the specification and claims of the present invention should not be interpreted as limited to its general or its literal meaning, and should be interpreted as definition and concepts corresponding to the technical idea of the present invention based on the principle that an inventor may appropriately define the notion of terms to describe the invention in the best way possible. Also, exemplary embodiments and configuration illustrated in the drawings described herein are only one of the preferred embodiments, and does not represent all of the technical ideas of the present invention, thus it should be understood that there are various equivalents and variations that may substitute these at the time of the present application.

Hereinafter, a method for manufacturing a fingerprint recognition home key using a transparent injection molding material and a UV curable bond in accordance with embodiments of the present invention is described in detail in reference to the accompanying drawings.

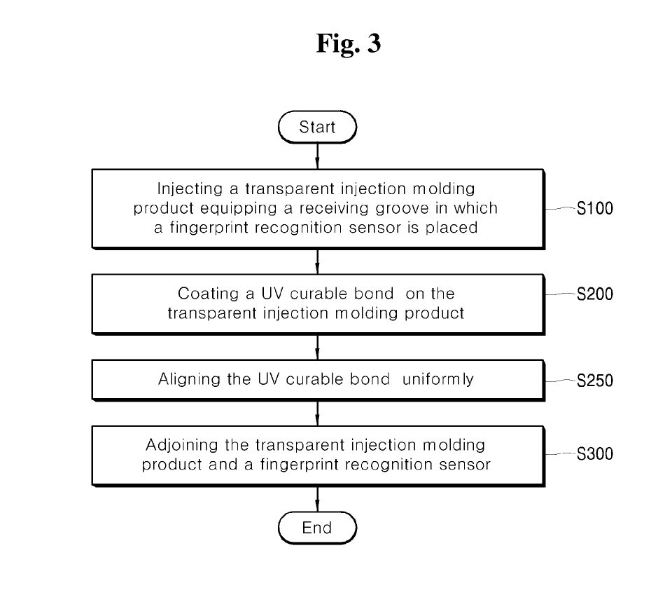

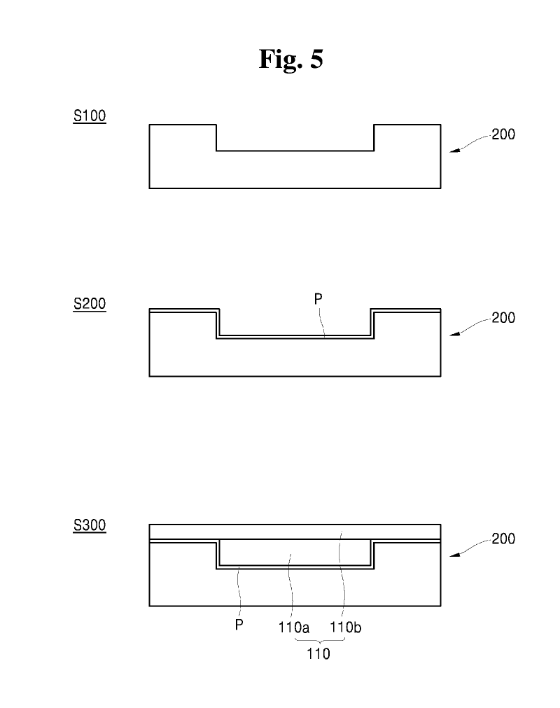

FIG. 3 is a flow chart of a method of manufacturing a fingerprint recognition home key for improving an attachment precision of a fingerprint recognition sensor in accordance with a first embodiment of the present invention, and FIG. 5 is a process chart thereof.

Therefore, with respect to describing each step in detail, it will be described by referring to the flow chart of FIG. 3 and the process chart of FIG. 5 together.

Injection molding transparent injection molding product (S100)

This step is an injection step in which a transparent injection molding product equipping a receiving groove on which a fingerprint recognition sensor is placed is molded.

Especially, it may be understood as a step molding the first injection molding product provided in a shape for fastening the fingerprint recognition sensor transparently.

That is, the first injection molding product may be a transparent injection molding product.

As illustrated in FIG. 5, the transparent injection molding product (200) is equipped with an receiving groove on top for the fingerprint recognition sensor to be placed and fastened, and the shape of this receiving groove does not need to be limited to the illustrated shapes and may be altered to a variety of shapes.

In this step, a transparent injection molding material may be used for injection molding the transparent injection molding product (200).

For a detailed example, an injection material composed of a transparent material comprising at least one among PC, PC glass, ABS, PP, PET, nylon, and plastic polymer compound may be used

Coating UV curable bond (S200)

This step is a step coating a UV curable bond on top of the first injection molding product (That is, a transparent injection molding product), which was transparently injection molded in the previous step.

And this step may further comprise uniformly aligning so that the coated UV curable bond may have an entirely uniform thickness (S250). Commonly known bonds in a liquefied form are only able to adjust the amount of supply, and had difficulties of maintaining a uniform coating state as it inclines to one side on a coated surface because the liquefied form itself has high liquidity.

But in accordance with the first injection molding product being provided in the shape of the transparent injection molding product in the previous step, the coating state of the coated bond on top of the transparent injection molding product may be identified through a worker (or a separate optical device), and the bond may be adjusted to be coated in a uniform thickness.

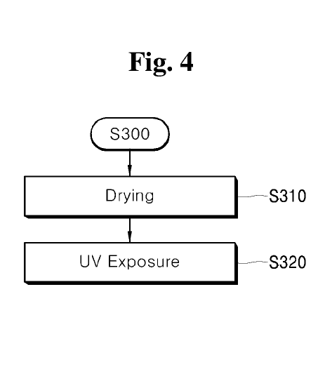

And the UV curable bond used in the present step, as shown in FIG. 4, since it may be cured into a solid state by going through a drying step (S310) and/or a UV exposure (S320), adhesion with each other may be strengthened when adjoining the fingerprint recognition sensor and the transparent injection molding product, which is the following step.

As illustrated in FIG. 5, when the transparent injection molding product (200) is provided after going through the first injection process in the previous step (S100), the UV curable bond (P) is entirely coated on top of this transparent injection molding product (200) in a uniform thickness. If a defective coating, in which the coated UV curable bond (P) inclines to any one way, appear, this may be improved through a bond alignment operation.

Accordingly, the attachment precision may be improved between the fingerprint recognition sensor and the transparent injection molding product.

This brings about the result of improving fingerprint recognition.

Adjoining a fingerprint recognition sensor and a transparent injection molding product (S300)

In the present step, the fingerprint recognition sensor is adjoined and fastened on top of the transparent injection molding product using the UV curable bond, which is coated on the transparent injection molding product in the previous step.

The fingerprint recognition sensor of the present step is a capacitive fingerprint recognition sensor, and illustrated in FIG. 5, the fingerprint recognition sensor (110) comprises an ASIC (110a) and a film (110b) (hereinafter, referred to as a film of the fingerprint recognition sensor).

The ASIC (110a) takes the role of changing the detected analog data to a digital signal and is electrically connected to the FPCB film (110b), on which a pattern where fingerprints are recognized is formed. The film of the fingerprint recognition sensor (110b) describes a FPCB formed with a PI film.

The fingerprint recognition sensor (110) is attached facing each other in a uneven joint shape through the previously coated UV curable bond (P) on top of the transparent injection molding product (200), and the attached state between each other is strongly maintained through the drying and/or the UV exposure of the UV curable bond .

In the case of the fingerprint recognition home key provided by this manufacturing method, the bond coated on the adjoining part between the fingerprint recognition sensor and the injection molding product may be entirely aligned in a uniform thickness, and especially, the alignment operation may become possible in accordance with the reason the injection molding product is transparent.

Furthermore, the attachment precision between each other may be improved in accordance with using the UV curable bond, and a fingerprint recognition ratio of 90% or over for the fingerprint recognition home key may be secured.

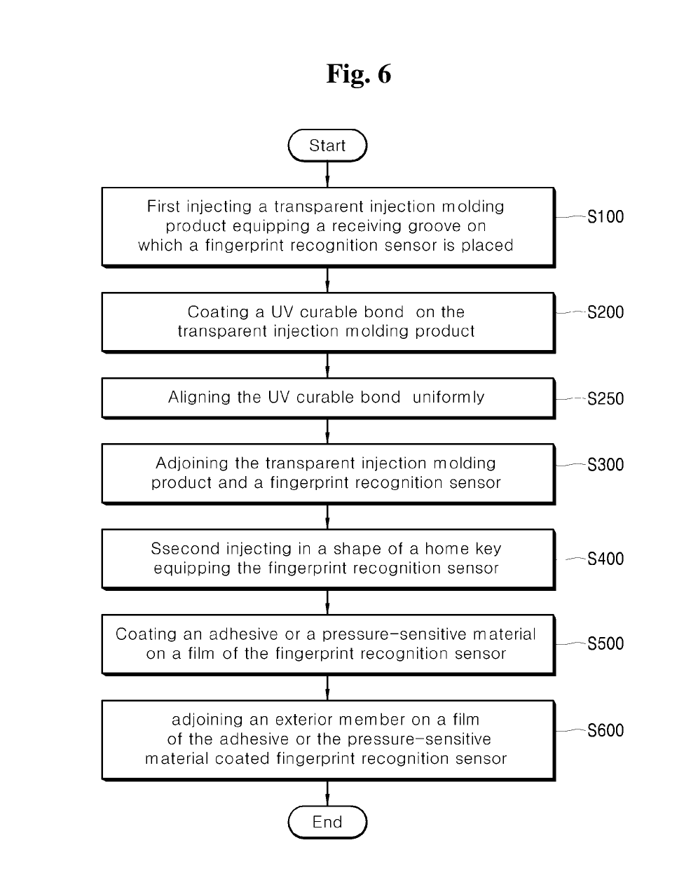

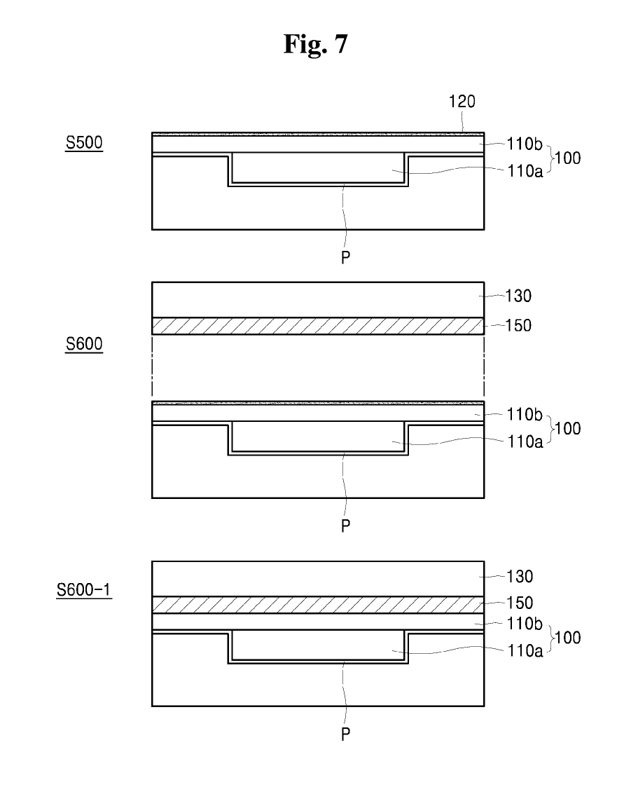

FIG. 6 is a flow chart of a method of manufacturing a fingerprint recognition home key for improving an attachment precision of a fingerprint recognition sensor in accordance with a second embodiment of the present invention, and FIG. 7 is an overall process chart thereof.

Referring to FIG. 6, performed following the entire steps (S100, S200, S300) in accordance with the first embodiment of the present invention examined through the previous FIG. 3 to FIG. 5, a second injection step (S400), coating an adhesive or a pressure-sensitive adhesive material (S500) and adjoining an exterior member (S600) may be comprised.

First, molding a second injection molding product in a home key shape using the first injection molding product on which the fingerprint recognition sensor is attached (S400) is comprised.

The present step is a step performed following the adjoining the fingerprint recognition sensor and the transparent injection molding product (S300) of the first embodiment of the present invention described above, and is a step in which second injecting the first injection molding product (that is, a transparent injection molding product), on which the fingerprint recognition sensor is attached using the UV curable bond, is carried out.

Here, the shape of the second injection molding product in the second injected home key shape is not separately illustrated, and this form may be provided in slightly different shapes for each embodiment. Next, coating an adhesive or a pressure-sensitive adhesive material (S500) on the film of the fingerprint recognition sensor is comprised.

As illustrated in FIG. 7, an adhesive or a pressure-sensitive adhesive material (120) is coated on top of the film of the fingerprint recognition sensor (110b). Here, the adhesive or the pressure-sensitive adhesive material (120) may use commonly known adhesives or pressure-sensitive adhesives such as bond, etc., and for a different form, a double-sided tape having adhesive or pressure-sensitive adhesive functions may be used.

When using the double-sided tape, it may be used by attaching to the top of the film of the fingerprint recognition sensor (110b). Also, when coating a liquefied adhesive or pressure-sensitive adhesive, a fixed quantity dispenser may be used. Next, adjoining a prepared exterior member on the film of the fingerprint recognition sensor, on which the adhesive or the pressure-sensitive adhesive material is previously coated, (S600) is comprised.

As illustrated in FIG. 7, an exterior member (130) is adjoined on top of the film of the fingerprint recognition sensor (110b), on which the adhesive or the pressure-sensitive adhesive material (120) is coated in the previous step (S500).

Then, as can be identified through the process chart of the step where adjoining of the exterior member is completed, a strong fastened state between the film of the fingerprint recognition sensor and the exterior member may be maintained.

A variety of materials having excellent permittivity may be used for the exterior member (130). For a detailed example, 3 types may be used for the material for the exterior member (130). First, a common glass or a reinforced glass may be used. Second, a sapphire glass may be used. Third, a plastic film or a thin film injection molding product may be used.

First, when the normal glass or the reinforced glass with excellent permittivity is used, having a thickness in the range of 40 to 100 nm is preferable.

If, in this case, the thickness of the exterior member is less than 40 nm, problems of damage or breaking of the exterior member from fingerprint contact due to the thickness being too thin may occur. On the contrary, if the thickness of the exterior member exceeds 100 nm, the fingerprint recognition may become poor as the distance between the fingerprint and the sensor become farther due to the thickness being too thick.

And when a sapphire glass having a more excellent permittivity than the normal glass or the reinforced glass is used, having a thickness in the range of 70 to 140 nm is preferable.

If the thickness of the exterior member is less than 70 nm, there are concerns of damage or breaking due to the thickness being too thin. On the contrary, if the thickness of the exterior member exceeds 140 nm, it is because the fingerprint recognition may degrade even if the permittivity is excellent compared to the normal glass or the reinforced glass

And when a plastic film or a thin film injection molding product is used for the exterior member, having a thickness in the range of 30 to 130 nm is preferable.

In this case, if the thickness of the exterior member is less than 30 nm, there are concerns of damage or breaking due to the thickness being too thin. On the contrary, if the thickness of the exterior member exceeds 130 nm, problems of the fingerprint recognition degrading may occur.

In the case of these exterior members, may have any shape among an entirely flat first shape, an upwardly protruding convex second shape, and a downward concave third shape.

When following prior coating methods, it could not have a second shape of a protruding convex or a third shape of a concave, but in the case of the present invention, a variety of exterior appearance as such may be bestowed in accordance with using materials of glass, etc. for a material of the exterior member.

Also, through the adjoining the exterior member step (S600), which is the present step, color printing may be performed on the back surface of the prepared exterior member since the prepared exterior member is adjoined on the film of the fingerprint recognition sensor.

Especially, a deposition for color printing may be further performed before the operation of printing colors on the back surface of the exterior member. For a detailed example, a print layer of black or white, etc. may be formed.

Also, the color printing operation may be performed at least once, and may be performed 2 times or more according to needs. The deposition and color print layer (150) equipped to the back surface of the exterior member (130) may have a thickness in the range of 2 to 30 nm.

So far, a method of manufacturing a fingerprint recognition home key for improving an attachment precision of a fingerprint recognition sensor in accordance with a preferred embodiment of the present invention was examined.

It should be understood that the exemplary embodiments described above is for exemplification only and is not limited thereto, and the scope of the present invention is defined only by the accompanying claims and equivalents thereof and not by the detailed description above. And it should be understood that along with the meaning and the range of the accompanying claims, all modifications and variations derived from equivalent concepts thereof is also within the scope of the present invention.

What is claimed is:

1. A method of manufacturing a fingerprint recognition home key for improving an attachment precision of a fingerprint recognition sensor comprising the steps of:
(a) an injection step in which a transparent injection molding product equipping a accommodating groove in which a fingerprint recognition sensor is placed is molded;
(b) coating a UV curable bond on the transparent injection molding product; and
(c) adjoining the transparent injection molding product and a fingerprint recognition sensor using the UV curable bond .

2. A method of manufacturing a fingerprint recognition home key for improving an attachment precision of a fingerprint recognition sensor according to claim 1, in the (a) step, using an injection molding material composed of a transparent material comprising at least one among PC, PC glass, ABS, PP, PET, nylon, and plastic polymer compound.

3. A method of manufacturing a fingerprint recognition home key for improving an attachment precision of a fingerprint recognition sensor according to claim 1, in the (b) step, further comprising aligning a coating state of the UV curable bond uniformly.

4. A method of manufacturing a fingerprint recognition home key for improving an attachment precision of a fingerprint recognition sensor according to claim 1, in the (c) step, further comprising,
(c-1) drying the UV curable bond ; and
(c-2) performing a UV exposure to the dried UV curable bond.

5. A method of manufacturing a fingerprint recognition home key for improving an attachment precision of a fingerprint recognition sensor comprising the steps of:
(a) a first injection step in which a transparent injection molding product equipping a accommodating groove in which a fingerprint recognition sensor is placed is molded;
(b) coating a UV curable bond on the transparent injection molding product;
(c) adjoining the transparent injection molding product and a fingerprint recognition sensor using the UV curable bond ;
(d) a second injection step in which a second injection molding product in a shape of a home key equipping the adjoined fingerprint recognition sensor;
(e) coating an adhesive or a pressure-sensitive material on a film of the fingerprint recognition sensor; and
(f) adjoining a prepared exterior member on a film of the adhesive or a pressure-sensitive material coated fingerprint recognition sensor.

6. A method of manufacturing a fingerprint recognition home key for improving an attachment precision of a fingerprint recognition sensor according to claim 5, in the (a) step, using an injection molding material composed of a transparent material comprising at least one among PC, PC glass, ABS, PP, PET, nylon, and plastic polymer compound.

7. A method of manufacturing a fingerprint recognition home key for improving an attachment precision of a fingerprint recognition sensor according to claim 5, in the (b) step, further comprising aligning a coating state of the UV curable bond uniformly.

8. A method of manufacturing a fingerprint recognition home key for improving an attachment precision of a fingerprint recognition sensor according to claim 5, in the (c) step, further comprising,
(c-1) drying the UV curable bond ; and
(c-2) performing a UV exposure to the dried UV curable bond.

9. A method of manufacturing a fingerprint recognition home key for improving an attachment precision of a fingerprint recognition sensor according to claim 5, in the (e) step, using a method in which a pressure-sensitive adhesive or an adhesive is coated or a double-sided tape is attached on the film of the fingerprint recognition sensor.

10. A method of manufacturing a fingerprint recognition home key for improving an attachment precision of a fingerprint recognition sensor according to claim 5, before the (f) step, further comprising printing selected colors at least once on a back side of the exterior member

11. A method of manufacturing a fingerprint recognition home key for improving an attachment precision of a fingerprint recognition sensor according to claim 5, wherein the exterior member is composed of at least one material among a normal glass, a sapphire glass, a plastic film or a thin film injection molding product.
